(19) United States
(12) Patent Application Publication
(10) Pub. No.: US 2022/0022842 A1
(43) Pub. Date: Jan. 27, 2022

(54) SYSTEM AND METHOD FOR MEASURING A QUANTITY OF BREAST MILK CONSUMED BY A BABY

(71) Applicants: Brittany MOLKENTHIN, Canterbury, CT (US); Jayme COATES, Canterbury, CT (US)

(72) Inventors: Brittany MOLKENTHIN, Canterbury, CT (US); Jayme COATES, Canterbury, CT (US)

(21) Appl. No.: 17/382,781

(22) Filed: Jul. 22, 2021

Related U.S. Application Data
(60) Provisional application No. 63/054,855, filed on Jul. 22, 2020.

(51) Int. Cl. A61B 8/08; A61B 8/14; A61B 8/00; G01F 22/00

(52) U.S. Cl. CPC A61B 8/0833; A61B 8/14; G06F 17/18; G01F 22/00; A61B 8/4209

(57) ABSTRACT

A system for determining a volume of breastmilk ingested by a baby, e.g., an infant, during a breastfeeding session includes an ultrasound source configured to transmit ultrasonic waves to a stomach of the baby; an ultrasound sensor configured to receive ultrasonic echoes generated by reflection of the ultrasonic waves from the stomach; and a processor configured to detect a boundary of the stomach and a level of a liquid surface within the stomach based on the received ultrasonic echoes, and determine a volume ingested into the stomach during the breastfeeding session based on the detected boundary of the stomach and the detected level of the liquid surface. Ultrasound measurements during a breastfeeding session are not affected by natural processes such as swallowing air, voiding during feedings, and spitting up, providing increased confidence to breastfeeding mothers. These measurements are also obtained non-invasively, providing minimal if any distraction to the feeding child.

### CROSS REFERENCE TO RELATED APPLICATION

This application claims the benefit of U.S. Provisional Application Ser. No. 63/054,855, filed Jul. 22, 2020, which is incorporated herein by reference in its entirety.

### FIELD OF THE TECHNOLOGY

The present technology relates to determining a volume ingested during a feeding. More specifically, this technology relates to determining a volume of breast milk ingested by an infant during a breastfeeding session.

### BACKGROUND

The number one barrier to breastfeeding is the question of, how does one know if the infant is receiving enough at feedings. According to the Surgeon General, nearly 50% of women stop breastfeeding within the first two weeks postpartum because of the apprehension of the amount of breast milk that the infant is receiving. Technology options to monitor whether an infant is receiving enough breast milk include: monitoring baby growth; monitoring baby's diapers; monitoring baby's mood; and evaluating baby's ability to latch and swallow.

These techniques do not give real time feedback and are subjective, often creating confusion and anxiety. New mothers and even trained professionals observe and monitor these indicators, but until there is a physical issue with the infant there is no real indication that unambiguously indicates action must be taken. Due to this uncertainty, mothers who do not trust these indicators will default to bottle feeding, where each feeding can be quantified. Even mothers who bottle-feed breast milk sense the lack of mother-child connection. Additionally, although breast pump technology has recently been improved, pumping is often more painful and inconvenient.

Studies have shown that there is a correlation between breastfeeding and the preventive measure it poses for infectious diseases. There is a substantial increase in hospitalizations for infants that were formula-fed versus infants that were breast-fed for respiratory tract infections. Through meta-analysis, infants that are formula-fed are shown to have a higher risk of sudden infant death syndrome than those that are breast-fed. Breastfeeding has also been shown to provide short-term protection against childhood obesity. Not only are there many benefits for the infant to be breast-fed, but also many for the mother. These benefits include decreased risk of premenopausal breast cancer, possible decreased risk of postmenopausal breast cancer, suppression of maternal infertility, rebalancing of postpartum hormonal changes, and possible protection from ovarian cancer.

Current systems give an estimate of breast milk intake with a large margin of error. Breast pumps, a large sector of the breastfeeding supplies market, also quantify the amount of breast milk that an infant can receive during feeding. Some disadvantages of breastfeeding pumps include that they interfere with the actual breastfeeding experience, there is increased risk of contamination, bacteria, and unsafe milk practices, and there are decreased benefits from the milk due to the freezing and thawing. There is also an increased risk of mastitis, nipple wounds, and trauma. The utilization of breast pumps does not decrease the risk of ovarian cancer, breast cancer, diabetes, and metabolic syndrome for the mother, unlike the act of breastfeeding.

Some attempts have been made to solve the problem of calculating breast milk intake. One of the newest technologies on the market is Momsense® (Momsense Ltd., Ramat Gan, Israel). MomSense is a patented technology on the market that assesses acoustics as the baby swallows. These acoustics are then converted to trackable feeding patterns. MomSense claims lactation experts have been able to recognize the sounds of audible swallowing during breastfeeding. Lactation experts can state if there is a confirmed latch and the infant is swallowing correctly during breastfeeding. However, research has shown that swallows alone only account for approximately fifty percent of the variation of breast milk intake. Additionally, that product incorporates adhesives that are required to adhere a wired microphone to the infant's cheek, which have been shown to interrupt the natural component of the breastfeeding process. Adhesives have limited use and require re-stocking. Additionally, that is a wired solution; the wire can easily be grabbed and distract an infant.

MilkSense measures the impedance of breast tissue before and after breastfeeding. The impedance change correlates to quantity of milk. The MilkSense product has no public data or test results to provide accuracy, but within an extensive user manual it identifies significant reliance on body position, electrode medium, and calibration for accurate measurements that are challenging for new mothers breastfeeding their hungry infants around the clock. Additionally, that product does not work with colostrum, which is the milk produced in the critical early days of breastfeeding, a critical time to establish breastfeeding and bonding with the infant. The MilkSense product requires calibration and must be used with a specialized solution added into the base unit regularly. The device requires mothers' full attention and two hands to perform the pre- and post-breastfeeding readings, so the baby needs to be set down in order to set up the device and perform the measurement prior to feeding.

HatchBaby correlates the change in a baby before and after a feeding with the associated weight change. Weighing infants in the clinical setting has also been a method utilized for quantifying breast milk intake. Research has shown that the commonly utilized weighing method lacks precision. In one study, 95% of cases deviated 15 mL, or nearly 40% of the infant's intake. That is a large error, especially when infant intake is in smaller amounts the first few weeks of life. That large margin of error would be a potential concern for providers that would want to quantify breast milk consumption in the healthcare setting. Some studies have demonstrated that weighing an infant could be an effective method of measuring breast milk intake. However, despite those positive findings, each study of weighing infants was performed under consistent, controlled methods by eliminating many variables that could cause error. Weighing an infant in that kind of controlled manner is unrealistic for mothers in the home setting.

### SUMMARY

Accordingly, some embodiments of the present disclosure relate to a system for determining a volume ingested during a feeding. The system includes an ultrasound source configured to transmit ultrasonic waves to a stomach; an ultrasound sensor configured to receive ultrasonic echoes generated by reflection of the ultrasonic waves from the stomach; and a processor configured to detect a boundary of the stomach and a level of a liquid surface within the stomach based on the received ultrasonic echoes, and determine a volume ingested into the stomach over a time period based on the detected boundary of the stomach and the detected level of the liquid surface.

In some embodiments, the stomach is a stomach of a human baby. In some embodiments, the volume ingested is a volume of breast milk. In some embodiments, the ultrasound source and the ultrasound sensor are positioned on a substrate. In some embodiments, the time period starts prior to a time of a start of the feeding or at the time of the start of the feeding. In some embodiments, the time period ends at a time of an end of the feeding or after the time of the end of the feeding. In some embodiments, the substrate is a garment. In some embodiments, the ultrasound source and the ultrasound sensor are positioned on the substrate so as to allow the ultrasound source and the ultrasound sensor to interface with skin proximate the stomach. In some embodiments, the ultrasonic waves are provided at a frequency of about 7 MHz. In some embodiments, the processor is further configured to determine the volume ingested into the stomach using the following formula: V=π/6*a*b*c, where a is a longitudinal radius of the stomach, b is a transverse radius of the stomach, and c is an antero-posterior radius of the stomach. In some embodiments, the formula further includes a correction factor to adjust for an age of a being having the stomach. In some embodiments, system further includes an indicator configured to indicate the determined volume ingested into the stomach. In some embodiments, the indicator includes a display, and the display is configured to display a numerical representation of the determined volume ingested into the stomach. In some embodiments, the indicator includes a plurality of LEDs, and the processor is further configured to illuminate a number of the LEDs corresponding to the determined volume ingested into the stomach.

Some embodiments of the present disclosure relate to another system for quantifying a volume ingested during feeding. The system includes an ultrasound transducer configured to transmit ultrasonic waves to a stomach, and receive ultrasonic echoes generated by reflection of the ultrasonic waves from the stomach; and a processor configured to detect a boundary of the stomach and a level of a liquid surface within the stomach based on the received ultrasonic echoes, and determine a volume ingested into the stomach over a time period based on the detected boundary of the stomach and the detected level of the liquid surface.

Some embodiments of the present disclosure relate to a method for quantifying a volume ingested during a feeding. The method includes transmitting ultrasound waves to a stomach; receiving ultrasonic echoes generated by reflection of the ultrasonic waves from the stomach; detecting a boundary of the stomach and a level of a liquid surface within the stomach based on the received ultrasonic echoes; and determining a volume ingested into the stomach over a time period based on the detected boundary of the stomach and the detected level of the liquid surface.

In some embodiments, the transmitting of the ultrasonic waves includes applying an ultrasound source to a surface of skin proximate the stomach such that the surface of the skin and the ultrasound source abut; and transmitting the ultrasonic waves from the ultrasound source to the surface of the skin proximate the stomach. In some embodiments, the determining of the volume comprises determining the volume using the following formula: V=π/6*a*b*c, where a is a longitudinal radius of the stomach, b is a transverse radius of the stomach, and c is an antero-posterior radius of the stomach. In some embodiments, the method further includes indicating the determined volume using a display or LEDs. In some embodiments, a non-transitory computer-readable medium stores instructions that, when executed by a processor, cause the processor to perform the method.

Additional details and feature of embodiments of the technology will now be described in connection with the following drawings.

### BRIEF DESCRIPTION OF THE DRAWINGS

The drawings show embodiments of the disclosed subject matter for the purpose of illustration. However, it should be understood that the present application is not limited to the precise arrangements and instrumentalities shown in the drawings, wherein:

FIG. 1A is a front view of a system for determining a volume of breast milk ingested during breastfeeding, according to an embodiment of the present disclosure.

FIG. 1B is a schematic representation of an ultrasound source, according to an embodiment of the present disclosure.

FIG. 1C is a schematic representation of a system for determining a volume of breast milk ingested during breastfeeding, according to an embodiment of the present disclosure.

FIG. 2 is a chart of a method for determining a volume of breast milk ingested during breastfeeding, according to an embodiment of the present disclosure.

### DETAILED DESCRIPTION

It is recognized that certain limitations and features described in the present disclosure, such as ultrasound sources or sensors, may need to be modified or removed in order to be in compliance with applicable local laws. By way of example, in the United States, certain limitations and features may need to be modified or removed in order to be in compliance with the Health Insurance Portability and Accountability Act (HIPAA).

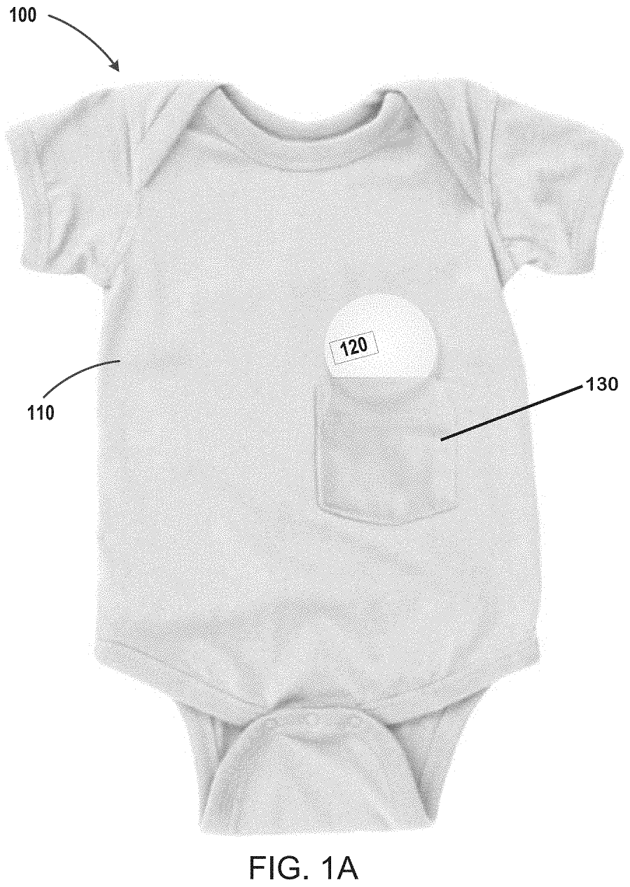

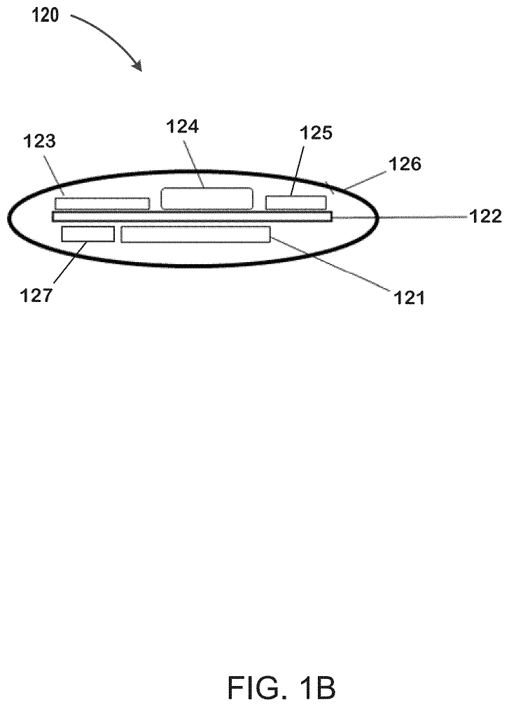

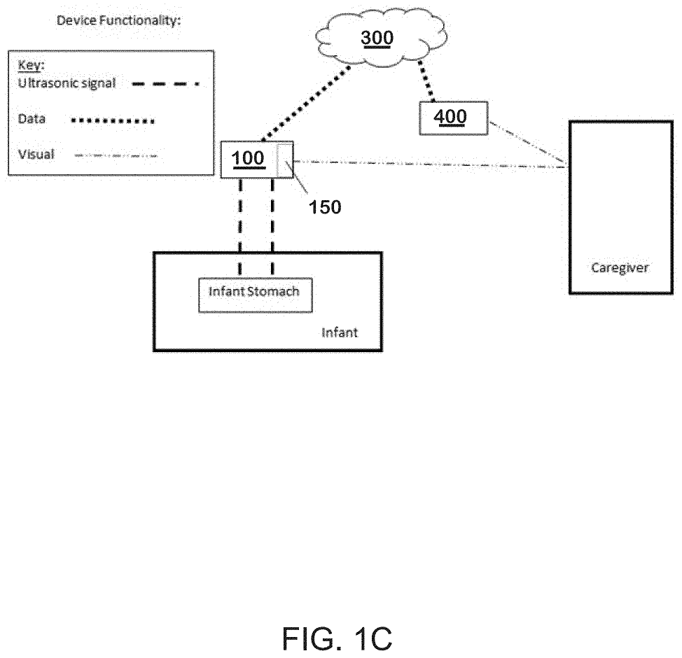

Referring now to FIG. 1A, some aspects of the disclosed subject matter are directed to a system 100 for quantifying a volume ingested by a human during a feeding. While the present disclosure shows and describes an exemplary embodiment of system 100 wherein the human is a baby and the volume ingested is a volume of breast milk, system 100 is not limited in this regard. As used herein, the term “feeding” is used to refer to the ingestion of milk or milk-like liquids and solids, e.g., breast milk.

Referring to FIG. 1A, in some embodiments, system 100 includes at least one substrate 110. The substrate 110 includes one or more surfaces configured to comfortably interface with human skin. In some embodiments, at least one of the surfaces interfaces substantially uninterruptedly with a surface of the human skin. In some embodiments, substrate 110 includes a disk, spheroid body, polyhedral body, bib, shirt, blouse, dress, frock, onesie, or a combination thereof. In some embodiments, the substrate 110 and/or the one or more substrate surfaces are composed of a biocompatible material. In some embodiments, the substrate 110 is a garment. In some embodiments, the garment is sized to fit a human baby and covers at least the torso of the human baby.

System 100 includes ultrasound source 120 configured to emit ultrasonic energy. In some embodiments, ultrasound source 120 is positioned on substrate 110. The ultrasound source 120 is configured to provide the ultrasound energy to the human stomach, e.g., the stomach tissues and the stomach contents. The ultrasound source 120 can be of any number, layout, and orientation so long as ultrasonic energy is delivered through the skin to the stomach. In some embodiments, the ultrasound source 120 includes multiple ultrasound sources arranged in a grid, a line, a circle, etc. In some embodiments, substrate 110 also includes one or more additional sensors, e.g., an infrared light sensor, a heart rate sensor, a respiratory sensor, a temperature sensor, or a combination thereof (not shown). In some embodiments, system 100 includes one or more infrared light sources (not shown).

In the embodiment of FIG. 1A, the substrate 110 is a onesie. The substrate 110 (i.e., the onesie) includes a pouch 130 sewn on the substrate 110 (i.e., the onesie). In some embodiments, the interior side of the pouch 130 includes a hole (not shown) to allow the ultrasound source 120 to come into direct contact with the skin proximate the stomach of the wearer of the onesie (e.g., a baby). In some embodiments, the pouch 130 is disposed at the center of the front of the substrate 110 (i.e., the onesie). In some embodiments, the pouch 130 is disposed on the centerline of the substrate 110 (i.e., the onesie). In other embodiments, the pouch 130 is disposed off-center (i.e., disposed to the left or to the right of the centerline of the onesie).

When in use, in some embodiments, substrate 110 and/or ultrasound source 120 is applied to or proximate to the surface of the skin proximate the stomach, such that the surface of the skin and ultrasound source 120 are abutting. In some embodiments, the ultrasonic energy from ultrasound source 120 is emitted substantially perpendicular to the surface of the skin. In some embodiments, the ultrasonic energy from ultrasound sources 120 is emitted at an angle with respect to the surface of the skin. In some embodiments, the ultrasonic energy from ultrasound source 120 is emitted substantially perpendicular to the surface of substrate 110 on which ultrasound source 120 is disposed. In some embodiments, the ultrasonic energy from ultrasound source 120 is oriented at an angle with respect to the surface of substrate 110 on which ultrasound sources 120 is disposed. In some embodiments, the ultrasonic energy from ultrasound source 120 is scanned over at least a portion of the stomach. In some embodiments, the ultrasound source 120 transmits ultrasonic waves to the stomach through the skin and through the tissue between the skin and the stomach.

Referring now to FIG. 1B, in some embodiments, ultrasound source 120 includes an ultrasonic transmitter 121. In some embodiments, ultrasound source 120 includes a printed circuit board 122. In some embodiments, ultrasound source 120 includes one or more processors and memory 123. In some embodiments, ultrasound source 120 includes one or more power sources 124, e.g., batteries. In some embodiments, ultrasound source 120 includes a communication module 125, e.g., a wireless communication apparatus to facilitate, for example, transmission of data via Bluetooth, Wi-Fi, etc. In some embodiments, the ultrasonic transmitter 121, printed circuit board 122, processors and memory 123, power source 124, and communication module 125 are contained within a housing 126.

In some embodiments, ultrasound source 120 includes an ultrasound sensor 127. In some embodiments, the ultrasound sensor 127 is remote from the ultrasound source 120. In some embodiments, the ultrasound sensor 127 is positioned on the same substrate 110 as ultrasound source 120. In some embodiments, the ultrasound sensor 127 is positioned on another substrate. In some embodiments, the ultrasound sensor 127 is positioned adjacent to the ultrasound source 120. In some embodiments, ultrasound source 120 and the ultrasound sensor 127 are positioned on the substrate 110 such that each interfaces with the skin proximate the human stomach. In some embodiments, the ultrasound sensor 127 is disposed inside the housing 126. Although in FIG. 1B the ultrasound emitter 121 and the ultrasound sensor 127 are shown as two separate elements, in some embodiments, the ultrasound emitter 121 and the ultrasound sensor 127 are the same element, i.e., an ultrasound transducer.

Without wishing to be bound by theory, the system 100 uses sound energy to calculate distances from a 3-D perspective. In some embodiments, ultrasonic energy is used to reflect boundaries between two structures on the stomach based on different acoustic resistances. In some embodiments, system 100 identifies the locations of the boundaries of the stomach, the level of liquid in the stomach, and their changes in 3-D space as the stomach fills with liquid, e.g., milk. In some embodiments, the ultrasound sensor 127 is configured to detect ultrasonic energy emitted by ultrasound source 120 that is reflected from the boundaries of the human stomach, as well as the liquid surface defining the liquid volume within the human stomach. In some embodiments, the volume is calculated via ellipsoid volume calculation according to formula I below:

$$V = \frac{π}{6} * a * b * c \qquad Formula I$$

wherein a=longitudinal radius of the stomach, b=transverse radius of the stomach, and c=antero-posterior radius of the stomach. The processor determines the longitudinal radius of the stomach, the transverse radius of the stomach, and the antero-posterior radius of the stomach based on a boundary of the stomach detected by the processor. In some embodiments, the overall infant stomach volume is detected according to Formula 1, where a=length of the stomach from the fundus to the antrum just prior to pylorus; b=distance from greater curvature to lesser curvature of the stomach; and c=depth of the body of the stomach. The processor determines the length of the stomach from the fundus to the antrum just prior to the pylorus, the distance from greater curvature to lesser curvature of the stomach, and the depth of the body of the stomach based on the boundary of the stomach detected by the processor. In some embodiments, a correction factor is included in Formula I. In some embodiments, the correction factor adjusts for the age of the human, e.g., an infant. In some embodiments, the “edge” of the milk (i.e., the level of milk within the stomach) is then detected and used to calculate how much of the stomach volume is filled with milk. In some embodiments, ultrasound source 120 is cycled on and off to obtain periodic readings of the milk content during a feeding. In some embodiments, ultrasound source 120 is pulsed.

In some embodiments, a first ultrasound reading is taken a time T=0 (i.e., a first instance) to define the baseline liquid volume in the stomach. That is, at T=0, the ultrasound source 120 transmits first ultrasonic waves to the stomach, and the ultrasound sensor 127 receives first ultrasonic echoes generated by reflection of the first ultrasonic waves from the stomach. The processor (e.g., a processor of the one or more processors and memory 123) generates a first estimate for a boundary of the stomach and a first estimate for a level of a liquid surface within the stomach based on the received first ultrasonic echoes. The processor then determines a first value for a volume of liquid in the stomach at T=0 based on the generated first estimate for the boundary of the stomach and the generate first estimate for the level of the liquid surface. In some embodiments, T=0 is a time of a start of feeding (e.g., breastfeeding). In other embodiments, T=0 is a time prior to the start of the feeding. In some embodiments, the stomach belongs to a baby.

In some embodiments, a subsequent ultrasound reading is taken at T=t. That is, at T=t, the ultrasound source 120 transmits second ultrasonic waves to the stomach, and the ultrasound sensor 127 receives second ultrasonic echoes generated by reflection of the second ultrasonic waves from the stomach. The processor generates a second estimate for the boundary of the stomach and a second estimate for the level of the liquid surface within the stomach based on the received second ultrasonic echoes. The processor then determines a second value for the volume of liquid in the stomach at T=t based on the generated second estimate for the boundary of the stomach and the generate second estimate for the level of the liquid surface. In some embodiments, T=t is a time of an end of the feeding. In other embodiments, T=t is after the time of the end of the feeding. In some embodiments, the second value for the volume of liquid in the stomach is greater than the first value for the volume of liquid in the stomach because a mother has breastfed the baby between T=0 and T=t. In other words, the liquid added to the stomach of the baby between T=0 and T=t is breast milk provided by the mother.

The processor then subtracts the first value for the volume of liquid in the stomach from the second value for the volume of liquid in the stomach to obtain the volume of liquid ingested into the stomach between T=0 and T=t. The volume ingested into the stomach is then indicated to the user. In some embodiments, the volume ingested into the stomach between T=0 and T=t is the volume of breast milk transferred from mother to baby during a breastfeeding session beginning at or after T=0 and ending at or before T=t.

In some embodiments, the volume of liquid is calculated repetitively to provide real-time or near-real-time information to the user about how much milk has been ingested since T=0. In those embodiments, the first value for the volume of liquid in the stomach at T=0 is determined as described above. The system 100 then waits for a predetermined interval until T=1. For example, the predetermined interval could be one decisecond, one second, one minute, two minutes, five minutes, ten minutes, or any other suitable time interval. At T=1, the ultrasound source 120 transmits second ultrasonic waves to the stomach, and the ultrasound sensor 127 receives second ultrasonic echoes generated by reflection of the second ultrasonic waves from the stomach. The processor generates a second estimate for the boundary of the stomach and a second estimate for the level of the liquid surface within the stomach based on the received second ultrasonic echoes. The processor then determines a second value for the volume of liquid in the stomach at T=1 based on the generated second estimate for the boundary of the stomach and the generated second estimate for the level of the liquid surface. The change in the volume of liquid in the stomach between T=0 and T=1 is the volume of liquid, e.g., breast milk, consumed between T=0 and T=1. Thus, the processor subtracts the first value for the volume of liquid in the stomach from the second value for the volume of liquid in the stomach to obtain the volume ingested into the stomach between T=0 and T=1. The volume of liquid ingested into the stomach between T=0 and T=1 is indicated to the user (e.g., by a reading on a display showing a numerical value for the volume of liquid ingested into the stomach between T=0 and T=1).

The processor then waits for the predetermined interval until T=2. For example, if the predetermined interval were one minute, the time between T=0 and T=1 would be one minute, the time between T=1 and T=2 would be one minute, and the time between T=0 and T=2 would be two minutes.

At T=2, the ultrasound source 120 transmits third ultrasonic waves to the stomach, and the ultrasound sensor 127 receives third ultrasonic echoes generated by reflection of the third ultrasonic waves from the stomach. The processor generates a third estimate for the boundary of the stomach and a third estimate for the level of the liquid surface within the stomach based on the received third ultrasonic echoes. The processor then determines a third value for the volume of liquid in the stomach at T=2 based on the generated third estimate for the boundary of the stomach and the generated third estimate for the level of the liquid surface. The change in liquid volume between T=0 and T=2 is the volume of liquid, e.g., breast milk, consumed between T=0 and T=2. In some embodiments, the processor subtracts the first value for the volume of liquid in the stomach from the third value for the volume of liquid in the stomach to obtain the volume of liquid ingested into the stomach between T=0 and T=2. In other embodiments, the processor subtracts the second value for the volume of liquid in the stomach from the third value for the volume of liquid in the stomach, and adds the resulting difference to the second value for the volume of the liquid in the stomach to obtain the volume of liquid ingested into the stomach between T=0 and T=2. The volume of liquid ingested into the stomach between T=0 and T=2 is then indicated to the user (e.g., the display is updated to display a numerical value of the volume of liquid ingested in the stomach between T=0 and T=2).

In some embodiments, the processor then waits for the predetermined interval until T=3. At T=3, the processor repeats the process described above to obtain the volume of liquid ingested into the stomach between T=0 and T=3. The processor then waits for the predetermined interval until T=4. At T=4, the processor repeats the process described above to obtain the volume of liquid ingested in the stomach between T=0 to T=4, and so on. Thus, the user is continuously apprised of the volume of liquid ingested into the stomach by the processor repetitively updating the displayed value of the volume of liquid ingested. In some embodiments, the system 100 enables the user to reset the time to T=0 before a next breastfeeding session so that the user can be provided with real-time or near-real-time information about how much milk was breastfeed to the baby during that next breastfeeding session.

Although in some embodiments presented herein the ultrasound source 120 transmits the ultrasonic waves and the ultrasound sensor 127 receives the ultrasonic echoes, this is not intended to be limiting. In some embodiments, the ultrasonic transmitter 121 of the ultrasound source 120 transmits the ultrasonic waves and the ultrasound sensor 127 of the ultrasound source 120 receives the ultrasonic waves. In some embodiments, an ultrasound transceiver transmits the ultrasonic waves and receives the ultrasonic echoes. In some embodiments, the ultrasound transceiver is part of the ultrasound source 120.

In some embodiments, the ultrasound parameter is in the range of 1-15 MHz. In some embodiments, the ultrasound frequency is 7 MHz, which is suitable for smaller structures and children due to better resolution. Additionally, in some embodiments, by using short pulse, the ultrasound sensor 127 will maximize axial resolution.

In some embodiments, the system and method determine the value for the volume ingested into the stomach without determining a volume of urine held by or expelled by the human having the stomach. In some embodiments, the system and method determine the value for the volume ingested into the stomach without determining a volume of liquid flowing into the mouth of the human having the stomach. In some embodiments, the system and method determine the value for the volume ingested into the stomach without using an infrared sensor. In some embodiments, the system and method determine the value for the volume ingested into the stomach without weighing the being having the stomach. In some embodiments, the system and method determine the value for the volume ingested into the stomach without using any sensor other an ultrasound sensor.

Referring now to FIG. 1C, in some embodiments, system 100 includes or makes use of a device 400 for communicating feeding data to a caregiver, e.g., an infant's mother. In some embodiments, device 400 is a computing device, e.g., smartphone, tablet, PDA, etc. The display of the device 400 is configured to display the volume ingested (e.g., the volume of breast milk ingested during a certain time period, as described above). In some embodiments, system 100 includes an indicator 150 that is configured to indicate the volume ingested. In some embodiments, the indicator 150 is part of the ultrasound source 120.

According to an embodiment, the system 100 transmits an ultrasonic signal through the stomach (e.g., an infant's stomach) via the ultrasound source 120, and the echo is reflected off of the infant's stomach and back to the system 100 where it is received by the ultrasonic sensor 127. The system 100 then uses the processor (e.g., the one or more processors and memory 123) to obtain the volume of milk ingested into the stomach according to the method described above.

In some embodiments, LEDs of the indicator 150 are illuminated to indicate the volume of milk ingested. In some embodiments, the number of LEDs illuminated corresponds to a volume of milk the system 100 has detected has been ingested since T=0; when the system 100 detects that a maximum recommended volume of milk has been ingested, all LEDs are illuminated. In other embodiments, the number of LEDs illuminated corresponds to the volume of liquid inside the stomach; when the system 100 detects that the stomach is filled to its maximum capacity, all LEDs are illuminated. In some embodiments, the LEDs are of multiple colors. In some embodiments, the brightness of the LEDs corresponds to the volume ingested. The user (e.g., the caregiver) can look at the LEDs to find out how much milk the infant has ingested and make a decision about whether to continue feeding the infant or stop feeding the infant. For example, when the user observes that the indicator 150 shows that the maximum recommended volume of milk has been ingested, the user stops the feeding of the infant.

In some embodiments, data of the volume of milk ingested is sent from the system 100 to a cloud 300. The cloud 300 holds the data until it is requested by the device 400. Upon receipt of the request for the data, the cloud 300 sends the data to the device 400. The device 400 then displays the data or prompts the user to open an app so that the data can be displayed. In other embodiments, the data is sent directly from the system 100 to the device 400 via Bluetooth, WiFi, or the like. In some embodiments, a status bar displayed on the device 400 is filled according to what volume of milk has been ingested during a feeding session. For example, when the system 100 detects that a maximum recommended volume of milk has been ingested, the status bar is completely filled. In some embodiments, a status bar displayed on the device 400 is filled according to the current volume of liquid in the stomach. For example, when the system 100 detects that the stomach is filled to its maximum capacity, the status bar is completely filled. The user (e.g., the caregiver) can look at the display of the device 400 to find out how much milk the infant has ingested and make a decision about whether to continue feeding the infant or stop feeding the infant. For example, when the user observes that the display of the device 400 shows that the maximum recommended volume of milk has been ingested, the user stops the feeding of the infant.

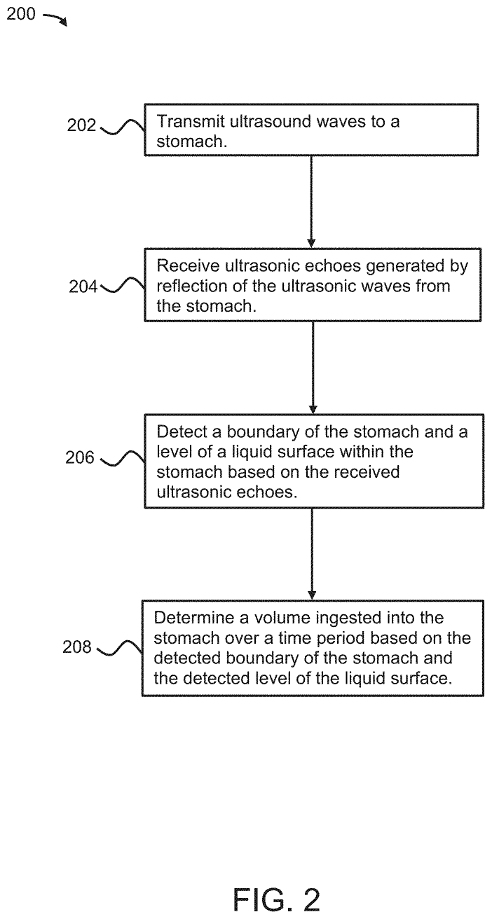

Referring to FIG. 2, a method 200 for quantifying a volume ingested by a human (e.g., a baby) during a feeding (e.g., breastfeeding) is provided. At step 202, ultrasound waves are transmitted to a stomach. In some embodiments, the ultrasound waves are transmitted by an ultrasound transmitter. In other embodiments, the ultrasound waves are transmitted by a transceiver. At step 204, ultrasonic echoes generated by reflection of the ultrasonic waves from the stomach are received. In some embodiments, the ultrasound echoes are received by an ultrasound sensor. In other embodiments, the ultrasound echoes are received by the ultrasound transceiver. At step 206, a boundary of the stomach and a level of a liquid surface within the stomach are detected based on the received ultrasonic echoes. In some embodiments, the boundary of the stomach and the level of the liquid surface within the stomach are detected by a processor. At step 208, a volume ingested into the stomach over a time period (e.g., a volume of milk ingested into the stomach of the baby during a breastfeeding session) is determined based on the detected boundary of the stomach and the detected level of the liquid surface. In some embodiments, the volume ingested is determined by a processor. In some embodiments, the processor causes the determined volume ingested to be indicated on a display, to be indicated by illuminating LEDs, or the like. In some embodiments, the processor causes the display, the LEDs, or the like to indicate that the volume ingested has reached a maximum recommended value when the processor detects that the volume ingested has reached the maximum recommended value. In some embodiments, the user (e.g., a caregiver) stops the feeding when the display, LEDs, or the like indicates that the volume ingested has reached the maximum recommended value.

The method and system of the present disclosure advantageously provide accurate measurements for the actual volume of milk ingested by a baby during a breastfeeding session. The device can indicate the volume visually and/or through cloud processing. For example, the caregiver's phone can display an indication of the volume of milk in the infant's stomach. This measurement is more accurate and more convenient than current technologies since it is a direct measurement of milk. Additionally, the device 400 is very convenient as it is wireless and does not require adhesives or additional consumables.

That ultrasound is not affected by natural processes such as swallowing air, voiding during feedings, and spitting up that occur during feedings, provides further confidence. The system and method are also truly non-invasive. There are no wires placed directly on the baby and there is no interference with the physical breastfeeding process, increasing comfort for both mom and baby. Finally, the system and method visualize the feeding in real-time, allowing parents to confirm whether or not a feeding is progressing successfully. Feeding data is also stored and tracked so that parents and other individuals, e.g., the baby's doctor, can view historical trends in the baby's feeding over time. The system and method of the present disclosure are particularly advantageous for premature babies, those born with complications, or those living in third world countries with limited resources.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. A system for determining a volume ingested during a feeding, comprising:
an ultrasound source configured to transmit ultrasonic waves to a stomach;
an ultrasound sensor configured to receive ultrasonic echoes generated by reflection of the ultrasonic waves from the stomach; and
a processor configured to detect a boundary of the stomach and a level of a liquid surface within the stomach based on the received ultrasonic echoes, and determine a volume ingested into the stomach over a time period based on the detected boundary of the stomach and the detected level of the liquid surface.

2. The system according to claim 1, wherein the stomach is a stomach of a human baby.

3. The system according to claim 2, wherein the volume ingested is a volume of breast milk.

4. The system according to claim 1, wherein the ultrasound source and the ultrasound sensor are positioned on a substrate.

5. The system according to claim 1, wherein the time period starts prior to a time of a start of the feeding or at the time of the start of the feeding.

6. The system according to claim 1, wherein the time period ends at a time of an end of the feeding or after the time of the end of the feeding.

7. The system according to claim 4, wherein the substrate is a garment.

8. The system according to claim 4, wherein the ultrasound source and the ultrasound sensor are positioned on the substrate so as to allow the ultrasound source and the ultrasound sensor to interface with skin proximate the stomach.

9. The system according to claim 1, wherein the ultrasonic waves are provided at a frequency of about 7 MHz.

10. The system according to claim 1, wherein the processor is further configured to determine the volume ingested into the stomach using the following formula:
$$V = \frac{π}{6} * a * b * c$$
wherein a is a longitudinal radius of the stomach, b is a transverse radius of the stomach, and c is an antero-posterior radius of the stomach.

11. The system according to claim 10, wherein the formula further includes a correction factor to adjust for an age of a being having the stomach.

12. The system according to claim 1, further comprising an indicator configured to indicate the determined volume ingested into the stomach.

13. The system according to claim 12, wherein the indicator includes a display, and the display is configured to display a numerical representation of the determined volume ingested into the stomach.

14. The system according to claim 12, wherein the indicator includes a plurality of LEDs, and the processor is further configured to illuminate a number of the LEDs corresponding to the determined volume ingested into the stomach.

15. A system for determining a volume ingested during a feeding, comprising:
an ultrasound transducer configured to transmit ultrasonic waves to a stomach, and receive ultrasonic echoes generated by reflection of the ultrasonic waves from the stomach; and
a processor configured to detect a boundary of the stomach and a level of a liquid surface within the stomach based on the received ultrasonic echoes, and determine a volume ingested into the stomach over a time period based on the detected boundary of the stomach and the detected level of the liquid surface.

16. A method for quantifying a volume ingested during a feeding, comprising:
transmitting ultrasound waves to a stomach;
receiving ultrasonic echoes generated by reflection of the ultrasonic waves from the stomach;
detecting a boundary of the stomach and a level of a liquid surface within the stomach based on the received ultrasonic echoes; and
determining a volume ingested into the stomach over a time period based on the detected boundary of the stomach and the detected level of the liquid surface.

17. The method according to claim 16, wherein the transmitting of the ultrasonic waves comprises:
applying an ultrasound source to a surface of skin proximate the stomach such that the surface of the skin and the ultrasound source abut; and
transmitting the ultrasonic waves from the ultrasound source to the surface of the skin proximate the stomach.

18. The method according to claim 16, wherein the determining of the volume comprises determining the volume using the following formula:
$$V = \frac{π}{6} * a * b * c$$
wherein a is a longitudinal radius of the stomach, b is a transverse radius of the stomach, and c is an antero-posterior radius of the stomach.

19. The method according to claim 16, further comprising indicating the determined volume using a display or LEDs.

20. A non-transitory computer-readable medium storing instructions that, when executed by a processor, cause the processor to perform the method according to claim 16.
